(19) United States
(12) Patent Application Publication
(10) Pub. No.: US 2026/0131043 A1
(43) Pub. Date: May 14, 2026

(54) LIQUID COMPOSITION FOR CONTACT LENS AND CONTACT LENS PRODUCT

(71) Applicant: PEGAVISION CORPORATION, TAOYUAN CITY (TW)

(72) Inventors: HAN-YI CHANG, TAOYUAN CITY (TW); CHUN-HAN CHEN, TAOYUAN CITY (TW); YEN-CHUN KO, TAOYUAN CITY (TW); YA-WEN WU, TAOYUAN CITY (TW); MIN-YUNG HUANG, TAOYUAN CITY (TW); WEI-AN YEH, Taoyuan City (TW)

(21) Appl. No.: 19/019,607

(22) Filed: Jan. 14, 2025

(30) Foreign Application Priority Data
Nov. 8, 2024 (TW) 113142834

(51) Int. Cl. A61L 12/14; A61L 12/10

(52) U.S. Cl. CPC A61L 12/14; A61L 12/10

(57) ABSTRACT

A liquid composition for contact lens and a contact lens product are provided, which include: a first hydrophilic component, a second hydrophilic component, an inorganic salt, and a liquid water. A content of the first hydrophilic component is not greater than 1 wt %. The first hydrophilic component is a polymer having the phosphorylcholine group, and the first hydrophilic component has a first weight-average molecular weight of not less than 4,000 Da. A content of the second hydrophilic component is not greater than 1 wt %. The second hydrophilic component is a hyaluronic acid or a salt thereof, and the second hydrophilic component has a second weight-average molecular weight of not greater than 10,000 Da.

### CROSS-REFERENCE TO RELATED PATENT APPLICATION

This application claims the benefit of priority to Taiwan Patent Application No. 113142834, filed on Nov. 8, 2024. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a liquid composition, and more particularly to a liquid composition for contact lens and a contact lens product.

### BACKGROUND OF THE DISCLOSURE

A conventional liquid composition for contact lens usually includes a hydrophilic component to improve a wettability of the contact lens. However, the conventional liquid composition for contact lens usually fails to effectively maintain a wettability of a surface of the contact lens for long-term wear, thereby causing a wearer to feel dry and uncomfortable. Although the liquid composition for contact lens currently on the market can provide a moisturizing effect to a certain extent in a short period of time through an addition of the hydrophilic component, the moisturizing effect gradually weakens over time and fails to meet a need for long-term wear. Furthermore, the conventional liquid composition for contact lens fails to provide with an effective combination of multiple components for simultaneously improving the wettability and having an effect of trapping water of the contact lens. Accordingly, an improved liquid composition for the contact lens that can durably maintain the wettability and comfort of the contact lens for long-term wear and reduce an occurrence of a problem of dry eye are in urgent need of development.

### SUMMARY OF THE DISCLOSURE

In response to the above-referenced technical inadequacies, the present disclosure provides a liquid composition for contact lens and a contact lens product.

In order to solve the above-mentioned problems, one of the technical aspects adopted by the present disclosure is to provide a liquid composition for contact lens. The liquid composition for contact lens includes a first hydrophilic component, a second hydrophilic component, an inorganic salt, and a liquid water. A content of the first hydrophilic component is not greater than 1 wt %, the first hydrophilic component is a polymer having a phosphorylcholine group, and the first hydrophilic component has a first weight-average molecular weight of not less than 4,000 Da. A content of the second hydrophilic component is not greater than 1 wt %, the second hydrophilic component is a hyaluronic acid or a salt thereof, and the second hydrophilic component has a second weight-average molecular weight of not greater than 10,000 Da. A content of the inorganic salt is not greater than 5 wt %. A content of the inorganic salt is not less than 80 wt %.

In one of the possible or preferred embodiments, the first hydrophilic component is selected from the group consisting of a homopolymer having the phosphorylcholine group and a copolymer having the phosphorylcholine group.

In one of the possible or preferred embodiments, the homopolymer having the phosphorylcholine group is poly(2-methacryloyloxyethyl phosphorylcholine), and the copolymer having the phosphorylcholine group formed by a copolymerization of 2-methacryloyloxyethyl phosphorylcholine and a methacrylic acid ester. The methacrylic acid ester is an alkyl methacrylate having a C2 to C12 alkyl group or a hydroxypropyl methacrylate having a C2 to C12 hydroxyalkyl group.

In one of the possible or preferred embodiments, the first weight-average molecular weight of the first hydrophilic component ranges from 4,000 Da to 1,000,000 Da, the second weight-average molecular weight of the second hydrophilic component is less than the first weight-average molecular weight, and the second weight-average molecular weight is not greater than 5,000 Da.

In one of the possible or preferred embodiments, the first weight-average molecular weight of the first hydrophilic component ranges from 100,000 Da to 1,000,000 Da, and the second weight-average molecular weight is not greater than 3,000 Da.

In one of the possible or preferred embodiments, the second hydrophilic component is a sodium salt of hyaluronic acid or a potassium salt of hyaluronic acid.

In one of the possible or preferred embodiments, the second hydrophilic component is the sodium salt of hyaluronic acid, and the second weight-average molecular weight of the second hydrophilic component is not greater than 1,000 Da.

In one of the possible or preferred embodiments, the second hydrophilic component is a sodium hyaluronate having the second weight-average molecular weight or a hydrolyzed sodium hyaluronate.

In one of the possible or preferred embodiments, based on a total weight of the liquid composition for contact lens being 100 wt %, a content of the first hydrophilic component ranges from 0.005 wt % to 1 wt %, a content of the second hydrophilic component ranges from 0.005 wt % to 1 wt %, a content of the inorganic salt ranges from 0.01 wt % to 5 wt %, and a content of the liquid water is not less than 90 wt %.

In one of the possible or preferred embodiments, the content of the first hydrophilic component ranges from 0.05 wt % to 1 wt %, and the content of the second hydrophilic component ranges from 0.01 wt % to 0.08 wt %.

In one of the possible or preferred embodiments, the first hydrophilic component has a first molality ranging from 1×10⁻⁸ mol/L to 2.5×10⁻⁴ mol/L, and the second hydrophilic component has a second molality ranging from 5×10⁻⁷ mol/L to 6×10⁻⁵ mol/L.

In one of the possible or preferred embodiments, the inorganic salt includes a chloride salt and a buffer salt, the chloride salt is selected from the group consisting of sodium chloride (NaCl), potassium chloride (KCl), and magnesium chloride (MgCl₂), and the buffer salt is selected from the group consisting of a boric acid or a salt thereof, a phosphoric acid or a salt thereof, a carbonic acid or a salt thereof, and citric acid or a salt thereof.

In order to solve the above-mentioned problems, another one of the technical aspects adopted by the present disclosure is to provide a contact lens product. The contact lens product includes a packaging container, the liquid composition for contact lens, and a contact lens soaked in the liquid composition for contact lens. The liquid composition for contact lens is filled in the packaging container. The contact lens is a hydrogel contact lens or a silicone hydrogel contact lens.

Therefore, in the liquid composition for contact lens and the contact lens product provided by the present disclosure, by virtue of “liquid composition for contact lens including a first hydrophilic component, a content of the first hydrophilic component being not greater than 1 wt %, the first hydrophilic component being a polymer having a phosphorylcholine group, and the first hydrophilic component having a first weight-average molecular weight of not less than 4,000 Da,” and “a content of the second hydrophilic component being not greater than 1 wt %, the second hydrophilic component being a hyaluronic acid or a salt thereof, and the second hydrophilic component having a second weight-average molecular weight of not greater than 10,000 Da,” a wettability of a surface of a contact lens can be greatly improved. In particularly, in the present disclosure, the liquid composition for contact lens can have a better wettability and a better moisturizing effect for trapping water of the surface of the contact lens than the conventional liquid composition for contact lens through an addition of the polymer having the phosphorylcholine group and a specific content of a low-molecular weight hyaluronic acid (or the salt thereof) in the liquid composition for contact lens.

These and other aspects of the present disclosure will become apparent from the following description of the embodiment taken in conjunction with the following drawings and their captions, although variations and modifications therein may be affected without departing from the spirit and scope of the novel concepts of the disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### First Embodiment

A first embodiment of the present disclosure provides a liquid composition for contact lens, the liquid composition for contact lens includes a first hydrophilic component (e.g., a first moisturizing component), a second hydrophilic component (e.g., a second moisturizing component), an inorganic salt, a liquid water, and selectively includes additives. The liquid composition for contact lens is especially suitable for being used as a storage solution, a disinfecting solution, or a cleaning solution of a contact lens.

The first hydrophilic component is a polymer having a phosphorylcholine group. In some embodiments of the present disclosure, the first hydrophilic component is selected from the group consisting of a homopolymer having the phosphorylcholine group and a copolymer having the phosphorylcholine group.

Specifically, the homopolymer having the phosphorylcholine group is poly(2-methacryloyloxyethyl phosphorylcholine) (i.e., poly-MPC), and the copolymer having the phosphorylcholine group is formed by a copolymerization of 2-methacryloyloxyethyl phosphorylcholine (MPC) and a methacrylic acid ester.

For example, the methacrylic acid ester can be an alkyl methacrylate or a hydroxypropyl methacrylate.

The alkyl methacrylate has a C2 to C12 alkyl group, and the alkyl methacrylate can be n-butyl methacrylate (BMA), 2-ethylhexyl methacrylate (EHMA), isodecyl methacrylate (IDMA), or lauryl methacrylate (LMA).

In addition, the hydroxypropyl methacrylate has a C2 to C12 hydroxyalkyl group, and the hydroxypropyl methacrylate can be 2-hydroxyethyl methacrylate (HEMA) or 2,3-dihydroxypropyl methacrylate (DHPM).

In the copolymer having the phosphorylcholine group, a number of moles of 2-methacryloyloxyethyl phosphorylcholine (MPC) is greater than a number of moles of the methacrylic acid ester, but the present disclosure is not limited thereto.

In one embodiment of the present disclosure, the first hydrophilic component (i.e., the polymer having the phosphorylcholine group) is a copolymer (i.e., poly(MPC-co-BMA) or PMB) of 2-methacryloyloxyethyl phosphorylcholine (MPC) and n-butyl methacrylate (BMA), but the present disclosure is not limited thereto.

For example, the first hydrophilic component (i.e., the polymer having the phosphorylcholine group) that is suitable for being used in the present disclosure can include, for example, poly(MPC-co-BMA), poly(MPC-co-EMHA), poly(MPC-co-IDMA), poly(MPC-co-LMA), poly(MPC-co-HEMA), poly(MPC-co-DHPM), etc.

Specifically, the first hydrophilic component has a first weight-average molecular weight. The first weight-average molecular weight (Mw1) of the first hydrophilic component (i.e., the polymer having the phosphorylcholine group) is not less than 4,000 Da (i.e., dalton), preferably ranges from 4,000 Da to 1,000,000 Da, and more preferably ranges from 100,000 Da to 1,000,000 Da.

It is worth mentioning that a molecular weight (i.e., weight-average molecular weight) mentioned in the present embodiment can be tested by, for example, gel permeation chromatography (GPC) according to ASTM D3593-80 testing standard, but the present disclosure is not limited thereto.

Based on a total weight of the liquid composition for contact lens being 100 wt %, a content of the first hydrophilic component (i.e., the polymer having the phosphorylcholine group) ranges from 0.005 wt % to 1 wt % (i.e., 50 ppm to 10,000 ppm), preferably ranges from 0.05 wt % to 1.0 wt % (i.e., 500 ppm to 10,000 ppm), and more preferably ranges from 0.1 wt % to 1.0 wt % (i.e., 1,000 ppm to 10,000 ppm), but the present disclosure is not limited thereto.

The second hydrophilic component is a hyaluronic acid or a salt thereof (i.e., a hyaluronic acid salt).

Specifically, the hyaluronic acid salt can be, for example, a sodium salt of hyaluronic acid (i.e., a sodium hyaluronate) or a potassium salt of hyaluronic acid (i.e., potassium hyaluronate).

Furthermore, the second hydrophilic component has a second weight-average molecular weight, and the second weight-average molecular weight is less than the first weight-average molecular weight of the first hydrophilic component. The second weight-average molecular weight (Mw2) of the second hydrophilic component (i.e., the hyaluronic acid or the hyaluronic acid salt) is not greater than 10,000 Da, preferably is not greater than 5,000 Da, more preferably is not greater than 3,000 Da, and the most preferably is not greater than 1,000 Da.

In a preferred embodiment of the present disclosure, the second hydrophilic component is the sodium salt of hyaluronic acid and is a low molecular weight sodium hyaluronate (i.e., having the second weight-average molecular weight) or a hydrolyzed sodium hyaluronate (e.g., HYBLOOM™ MINTURE or OLIGOHYAFERRE®), but the present disclosure is not limited thereto. Specifically, the hydrolyzed sodium hyaluronate is formed by hydrolyzing the sodium hyaluronate and is different from the sodium hyaluronate. Moreover, a hydrolysis process of the sodium hyaluronate relates an introduction of water and enzyme.

Based on the total weight of the liquid composition for contact lens being 100 wt %, a content of the second hydrophilic component (i.e., the hyaluronic acid or the hyaluronic acid salt) ranges from 0.005 wt % to 1.0 wt % (i.e., 50 ppm to 10,000 ppm), preferably ranges from 0.01 wt % to 0.08 wt % (i.e., 100 ppm to 800 ppm), and more preferably ranges from 0.01 wt % to 0.06 wt % (i.e., 100 ppm to 600 ppm), but the present disclosure is not limited thereto.

In one embodiment of the present disclosure, a first molality of the first hydrophilic component (i.e., the polymer having the phosphorylcholine group) ranges from 1×10⁻⁸ mol/L to 2.5×10⁻⁴ mol/L, and a second molality of the second hydrophilic component (i.e., the hyaluronic acid or the hyaluronic acid salt) ranges from 5×10⁻⁷ mol/L to 6×10⁻⁵ mol/L.

The inorganic salt can include, for example, a chloride salt and a buffer salt.

Specifically, the chloride salt can be selected from the group consisting of sodium chloride (NaCl), potassium chloride (KCl), and magnesium chloride (MgCl₂). The chloride salt is preferably sodium chloride (NaCl), and the chloride salt can help adjust an osmotic pressure of the storage solution of the contact lens to close to a normal tear osmolarity of an eye, thereby reducing discomfort when wearing the contact lens. Furthermore, the chloride salt can simulate an electrolyte balance of tears so as to maintain a normal physiological state of a surface of the eye. Moreover, a content of the chloride salt ranges from 0.01 wt % to 5 wt %.

The buffer salt is configured to adjust a pH value of the storage solution of the contact lens.

Specifically, the buffer salt can be selected from the group consisting of a boric acid or a salt thereof, a phosphoric acid or a salt thereof, a carbonic acid or a salt thereof, and citric acid or a salt thereof. Preferably, the buffer salt can be selected from the group consisting of the boric acid or the salt thereof and the phosphoric acid or the salt thereof.

The boric acid or the salt thereof can be selected from the group consisting of boric acid, sodium borate, and sodium tetraborate. Furthermore, the phosphoric acid or the salt thereof can be selected from the group consisting of sodium dihydrogen phosphate (NaH₂PO₄), disodium hydrogen phosphate (Na₂HPO₄), potassium dihydrogen phosphate (KH₂PO₄), and dipotassium hydrogen phosphate (K₂HPO₄).

Based on the total weight of the liquid composition for contact lens being 100 wt %, a content of the inorganic salt ranges from 0.01 wt % to 5 wt %.

The liquid water is a solvent (i.e. a balancing component) of the liquid composition for contact lens. Moreover, based on the total weight of the liquid composition for contact lens being 100 wt %, a content of the liquid water is not less than 80 wt %, preferably not less than 85 wt %, and more preferably is not less than 90 wt %.

It is worth mentioning that a pH value of the liquid composition for contact lens preferably ranges from 6 to 8, and an osmotic pressure of the liquid composition for contact lens preferably ranges from 250 osmol/Kg to 450 osmol/Kg.

In some embodiments of the present disclosure, the liquid composition for contact lens can further include a trace amount of the additives according to requirements. Specifically, the additives includes a third hydrophilic component (e.g., a third moisturizing component), and the third hydrophilic component can include, for example, polyvinyl alcohol (PVA), polyethylene glycol (PEG), a cellulose or a derivative thereof. The cellulose or the derivative thereof can be, for example, hydroxypropylmethylcellulose (HPMC), carboxymethyl cellulose (CMC), Methylcellulose (MC), hydroxy ethyl cellulose (HEC), or a cationic cellulose derivative.

Moreover, the liquid composition for contact lens can further include a surfactant (e.g., DLS, SLL, SDS, SBFA 30, TWEEN 20, or TWEEN 80) and/or a vitamin (e.g., vitamin B or vitamin C), and is not limited by the present disclosure.

### Second Embodiment

A second embodiment of the present disclosure provides a contact lens product including a packaging container, the above-mentioned liquid composition for contact lens, and a contact lens.

For example, the packaging container can be a plastic cup, such as a polypropylene (PP) plastic cup, and the packaging container has an accommodating space.

The liquid composition for contact lens is filled in the accommodating space of the packaging container.

The liquid composition for contact lens includes a first hydrophilic component, a second hydrophilic component, an inorganic salt, a liquid water, and selectively includes additives. It should be noted that a material characteristic of each of components in the liquid composition for contact lens and a proportion of a content of the liquid composition for contact lens are a same as those of the aforementioned first embodiment, and would not be further described herein for the sake of brevity.

The contact lens is placed in the accommodating space of the packaging container and is soaked in the liquid composition for contact lens. Specifically, the contact lens and the liquid composition for contact lens are jointly packaged in the packaging container, and the liquid composition for contact lens is configured to moist the contact lens and improve a wettability of a surface of the contact lens.

In some embodiments of the present disclosure, the contact lens can be, for example, a hydrogel lens or a silicone hydrogel lens.

A diameter (e.g., the unit is mm) of the contact lens can be represented as Dia, a center base curve (mm) of the contact lens can be represented as BC, a water content of the contact lens can be represented as WC %, and each of the aforementioned parameters needs to meet the following conditions: (1) 13.0<Dia<15.0, (2) 7.0<BC<9.0, and (3) 30.0%<WC %<70.0% (preferably ranges from 38% to 58%).

Specifically, the contact lens can be formed by, for example, curing a lens composition, and the lens composition includes a hydrophilic monomer (e.g., NVP, HEMA, MAA, or DMA, etc.), a crosslinking agent, an initiator (e.g., photoinitiator or thermal initiator), and selectively includes a tint and a siloxane monomer (e.g., a composition of the silicone hydrogel lens includes the siloxane monomer).

### [Experimental Data and Test Results]

The following experimental data is configured to describe a specific aspect of the present disclosure in detail, so that a person having an ordinary skill in the art can implement the present disclosure. However, the scope of the present disclosure is not limited to the following experimental data.

The following experiments are carried out by selectively adding a hyaluronic acid having a different molecular weight and a polymer having a phosphorylcholine group in a buffered saline solution to perform relevant tests.

A storage solution 1 includes: 0.708 wt % of sodium chloride, 0.470 wt % of boric acid, 0.05 wt % of sodium borate, and a solvent (i.e., the balancing component) that is water, and the storage solution 1 doesn't include the hyaluronic acid and the polymer having the phosphorylcholine group. That is, the storage solution 1 is a control group.

A storage solution 2 includes: 0.708 wt % of sodium chloride, 0.470 wt % of boric acid, 0.05 wt % of sodium borate, 0.06 wt % of a high-molecular weight hyaluronic acid having a weight-average molecular weight (Mw) that ranges from 80 k Da to 120 k Da (e.g., a sodium hyaluronate having a molecular weight of 100 kDa), and the solvent (i.e., the balancing component) that is water, and the storage solution 2 doesn't include the polymer having the phosphorylcholine group. That is, the storage solution 2 is a comparison group.

A storage solution 3 includes: 0.708 wt % of sodium chloride, 0.470 wt % of boric acid, 0.05 wt % of sodium borate, 0.06 wt % (i.e., 600 ppm) of a low-molecular weight hyaluronic acid having a weight-average molecular weight (Mw) of less than 1,000 Da (e.g., a sodium hyaluronate having a molecular weight of 900 Da from HYBLOOM™ MINTURE), and the solvent (i.e., the balancing component) that is water, and the storage solution 3 doesn't include the polymer having the phosphorylcholine group. That is, the storage solution 3 is the comparison group.

A storage solution 4 includes: 0.708 wt % of sodium chloride, 0.470 wt % of boric acid, 0.05 wt % of sodium borate, 0.06 wt % (i.e., 600 ppm) of the low-molecular weight hyaluronic acid having the weight-average molecular weight (Mw) of less than 1,000 Da (e.g., the sodium hyaluronate having the molecular weight of 900 Da from HYBLOOM™ MINTURE), 0.1 wt % (i.e., 1000 ppm) of the polymer having the phosphorylcholine group (i.e., poly(MPC-co-BMA) having a weight-average molecular weight that is approximately 550,000 Da and a molar ratio of MPC to BMA in poly(MPC-co-BMA) is approximately 3:1), and the solvent (i.e., the balancing component) that is water. That is, the storage solution 4 is an experimental group of the present disclosure.

A storage solution 5 includes: 0.708 wt % of sodium chloride, 0.470 wt % of boric acid, 0.05 wt % of sodium borate, 0.06 wt % (i.e., 600 ppm) of an ultra-high molecular weight hyaluronic acid having a weight-average molecular weight (Mw) that ranges from 850 k Da to 1,600 kDa (e.g., a sodium hyaluronate having a molecular weight of 1,225 k Da), 0.1 wt % (i.e., 1,000 ppm) of the polymer having the phosphorylcholine group (i.e., poly(MPC-co-BMA using in the storage solution 4), and the solvent (i.e., the balancing component) that is water. That is, the storage solution 5 is the comparison group.

Addition conditions of the hyaluronic acid and the polymer having the phosphorylcholine group in the aforementioned storage solutions 1 to 5 are summarized in Table 1 as follows.

### [Table 1].

| | Added Amount in the Buffered Saline Solution (ppm) | | | |
|---|---|---|---|---|
| | Low- | | | |
| | molecular | High- | Ultra-high | |
| | weight | molecular | molecular | |
| | hyaluronic | weight | weight | poly |
| | acid | hyaluronic | hyaluronic | (MPC-co- |
| | hyaluronic | acid Mw | acid Mw | BMA) |
| Groups of | acid Mw < | 80-120k | 850-1600k | (i.e., |
| Experimental | 1,000 Da | Da | Da | PMB) |
| Storage solution 1 | — (No additional hydrophilic components added) | | | |
| (control group) | | | | |
| Storage solution 2 | — | 600 ppm | — | — |
| (comparison | | | | |
| group) | | | | |
| Storage solution 3 | 600 ppm | — | — | — |
| (comparison | | | | |
| group) | | | | |
| Storage solution 4 | 600 ppm | — | — | 1,000 ppm |
| (experimental | | | | |
| group) | | | | |
| Storage solution 5 | — | — | 600 ppm | 1,000 ppm |
| (comparison | | | | |
| group) | | | | |

Next, each of the aforementioned storage solutions 1 to 5 is prepared to soak a hydrogel contact lens having a water content of 38%, a hydrogel contact lens having a water content of 58%, or a silicone hydrogel contact lens having a water content of 50%.

A time for soaking the aforementioned contact lenses (i.e., the hydrogel contact lens having a water content of 38%, the hydrogel contact lens having a water content of 58%, and the silicone hydrogel contact lens having a water content of 50%) is about 12 hours, and a temperature for soaking the aforementioned contact lenses is in normal temperature (e.g., 25° C.).

Then, the aforementioned contact lenses are tested by wettability tests including a dynamic contact angle (DCA), a weight loss, and a breaking-up time (BUT) after soaking. Relevant test methods and test results are described as follows:

<Dynamic Contact Angle (DCA)>: the dynamic contact angle of the aforementioned contact lenses are carried out by using a dynamic contact angle analyzer and measuring the dynamic contact angle of the aforementioned contact lenses by a captive bubble method, thereby obtaining a value of the dynamic contact angle (degree°). Specifically, the lower value of the dynamic contact angle indicates the better wettability of the surface of the contact lens.

The following Table 2 shows test results of the dynamic contact angle, which lists the test results of the dynamic contact angle of each of lens groups including the hydrogel contact lens having the water content of 38%, the hydrogel contact lens having the water content of 58%, and the silicone hydrogel contact lens having the water content of 50% that is respectively soaked in and taken out from the storage solutions 1 to 5.

### [Table 2].

| | Storage | Storage | Storage | Storage | Storage |
|---|---|---|---|---|---|
| Test Results | solution 1 | solution 2 | solution 3 | solution 4 | solution 5 |
| of Dynamic | (control | (comparison | (comparison | (experimental | (comparison |
| Contact Angle | group) | group) | group) | group) | group) |
| Hydrogel contact | 32.54° | 31.60° | 29.76° | 26.27° | 28.57° |
| lens having a water | | | | (preferably) | |
| content of 38% | | | | | |
| Hydrogel contact | 30.86° | 20.87° | 28.75° | 21.51° | 24.66° |
| lens having a water | | | | (preferably) | |
| content of 58% | | | | | |
| silicone hydrogel | 73.77° | 52.74° | 50.39° | 1.38° | 36.27° |
| contact lens having a | | | | (more | |
| water content of 50% | | | | preferably) | |

As shown in Table 2, the storage solution 4 (i.e., the experimental group, which includes 600 ppm of the low-molecular weight hyaluronic acid and 1,000 ppm of PMB) can enable the aforementioned three contact lenses (i.e., one of the lens groups) to have the lowest dynamic contact angle. That is, the storage solution 4 can enable a surface of each of the aforementioned three contact lenses to have the best wettability. It is worth mentioning that the storage solution 4 generates more significant performance on the silicone hydrogel contact lens, which decreases at least 10 degrees of the dynamic contact angle (e.g., decreases not greater than 5 degrees from a degree ranging from 36.12° to 73.77°) compared to other groups of storage solutions (i.e., the storage solution 1, 2, 3, and 5).

That is, the aforementioned experiments show that the dynamic contact angle of the contact lens can be decreased by adding hyaluronic acid having different molecular weights into the storage solution. Especially, under a condition that the storage solution includes PMB, an addition of the low-molecular weight hyaluronic acid having the Mw of less than 10,000 Da (i.e., the storage solution 4) has a better performance on the dynamic contact angle (DCA) of the contact lens to have a better wettability of the surface of the contact lens than an addition of the ultra-high molecular weight hyaluronic acid having the Mw that ranges from 850 k Da to 1600 k Da. In addition, the storage solution 4 generates more significant performance on the silicone hydrogel contact lens.

<Weight Loss>: the weight loss is carried out by taking the contact lens out from the storage solution to place in an external environment (e.g., at 25° C. and a relative humidity (RH) of 50%), and observing the weight loss over time (e.g., 15 minutes or 30 minutes) of the contact lens. The weight loss is calculated as follows:

Weight loss: (Original weight W1 of the contact lens taken out of the storage solution−Weight W2 of the contact lens after a predetermined time)/(Original weight W1 of the contact lens taken out of the storage solution)×100%.

The following Table 3-1 shows test results of a weight loss of the hydrogel contact lens having the water content of 38% of each of the lens groups after being taken out from the storage solutions 1 to 5.

[Table 3-1] The weight loss of the hydrogel contact lens having the water content of 38% of each of the lens groups.

| | Storage | Storage | Storage | Storage | Storage |
|---|---|---|---|---|---|
| | solution 1 | solution 2 | solution 3 | solution 4 | solution 5 |
| | (control | (comparison | (comparison | (experimental | (comparison |
| Weight Loss | group) | group) | group) | group) | group) |
| 15 minutes | 17.57% | 16.93% | 18.41% | 13.86% | 14.78% |
| | | | | (the lowest | |
| | | | | weight loss) | |
| 30 minutes | 28.70% | 28.32% | 29.59% | 24.93% | 27.22% |
| | | | | (the lowest | |
| | | | | weight loss) | |

As shown in Table 3-1, the hydrogel contact lens having the water content of 38% that is soaked in the storage solution 4 has the lowest weight loss (i.e., only 13.86% is lost in 15 minutes, and only 24.93% is lost in 30 minutes). Accordingly, the storage solution 4 enables the hydrogel contact lens having the water content of 38% to have the best moisturizing effect.

The following Table 3-2 shows test results of a weight loss of the hydrogel contact lens having a water content of 58% of each of the lens groups after being taken out from the storage solutions 1 to 5.

[Table 3-2] The weight loss of the hydrogel contact lens having the water content of 58% of each of the lens groups.

| | Storage | Storage | Storage | Storage | Storage |
|---|---|---|---|---|---|
| | solution 1 | solution 2 | solution 3 | solution 4 | solution 5 |
| | (control | (comparison | (comparison | (experimental | (comparison |
| Weight Loss | group) | group) | group) | group) | group) |
| 15 minutes | 15.08% | 13.89% | 13.59% | 12.38% | 13.43% |
| | | | | (the lowest | |
| | | | | weight loss) | |
| 30 minutes | 29.35% | 28.02% | 26.02% | 24.53% | 25.23% |
| | | | | (the lowest | |
| | | | | weight loss) | |

As shown in Table 3-2, the hydrogel contact lens having the water content of 58% that is soaked in the storage solution 4 has the lowest weight loss (e.g., only 12.38% is lost in 15 minutes, and only 24.53% is lost in 30 minutes). Accordingly, the storage solution 4 enables the hydrogel contact lens having the water content of 58% to have the best moisturizing effect.

The following Table 3-3 shows test results of a weight loss of the silicone hydrogel contact lens having a water content of 50% of each of the lens groups after being taken out from the storage solutions 1 to 5.

[Table 3-3] The weight loss of the silicone hydrogel contact lens having the water content of 50% of each of the lens groups.

| | Storage | Storage | Storage | Storage | Storage |
|---|---|---|---|---|---|
| | solution 1 | solution 2 | solution 3 | solution 4 | solution 5 |
| | (control | (comparison | (comparison | (experimental | (comparison |
| Weight Loss | group) | group) | group) | group) | group) |
| 15 minutes | 39.08% | 36.28% | 34.45% | 16.08% | 19.27% |
| | | | | (the lowest | |
| | | | | weight loss) | |
| 30 minutes | 45.23% | 44.23% | 43.09% | 33.72% | 37.81% |
| | | | | (the lowest | |
| | | | | weight loss) | |

As shown in Table 3-3, the silicone hydrogel contact lens having the water content of 50% that is soaked in the storage solution 4 has the lowest weight loss (e.g., only 16.08% is lost in 15 minutes, and only 33.72% is lost in 30 minutes). Accordingly, the storage solution 4 enables the silicone hydrogel contact lens having the water content of 50% to have the best moisturizing effect. It is worth mentioning that the storage solution 4 has a more significant moisturizing effect for trapping water on the silicone hydrogel contact lens, which can improve a wettability of a hydrophobic surface of the silicone contact lens.

<Breaking-up Time (BUT)>: the breaking-up time is carried out by using a photographic equipment to observe a time from the contact lens being taken out from the storage solution and being placed in the external environment until a water film of the contact lens breaks. Specifically, the unit of the time is second(s).

The following Table 4 shows test results of a breaking-up time of the hydrogel contact lens having the water content of 38%, the hydrogel contact lens having the water content of 58%, and the silicone hydrogel contact lens having the water content of 50% of each of the lens groups that are respectively soaked in and then taken out from the storage solutions 1 to 5.

### [Table 4].

| | Storage | Storage | Storage | Storage | Storage |
|---|---|---|---|---|---|
| | solution 1 | solution 2 | solution 3 | solution 4 | solution 5 |
| Breaking-up | (control | (comparison | (comparison | (experimental | (comparison |
| Time (sec) | group) | group) | group) | group) | group) |
| Hydrogel contact | 72 s | 86 s | 84 s | 97 s | 88 s |
| lens having the water | | | | (lasts the | |
| content of 38% | | | | longest) | |
| Hydrogel contact | 74 s | 87 s | 76 s | 107 s | 93 s |
| lens having the water | | | | (lasts the | |
| content of 58% | | | | longest) | |
| Silicone hydrogel | 30 s | 43 s | 31 s | 78 s | 59 s |
| contact lens having | | | | (lasts the | |
| the water content | | | | longest) | |
| of 50% | | | | | |

As shown in Table 4, each of the aforementioned three contact lenses that is soaked in the storage solution 4 has the longest breaking-up time (BUT), the hydrogel contact lens having the water content of 38% has the breaking-up time of 97 seconds, the hydrogel contact lens having the water content of 58% has the breaking-up time of 107 seconds, and the silicone hydrogel contact lens having the water content of 50% has the breaking-up time of 78 seconds. Accordingly, each of the aforementioned three contact lenses soaked in the storage solution 4 has the best moisturizing effect for trapping water. That is, the wettability of the contact lens can be effectively improved by adding the low-molecular weight hyaluronic acid having the molecular weight of less than 1,000 Da and PMB at a same time.

In conclusion, the low-molecular weight hyaluronic acid has a function of water storage, and PMB having a high-molecular weight has a function of trapping water. The low-molecular weight hyaluronic acid brings moisture into the contact lens, and PMB having the high-molecular weight forms a moisturizing film (i.e., water film) on an outer layer of the contact lens to trap moisture, thereby reducing the moisture loss from the contact lens. Accordingly, the water film of the outer layer of the contact lens can be more durable, thereby reducing a problem of dry eye caused by a long-term wear of the contact lens. Therefore, a performance of the breaking-up time of the contact lens in the storage solution 4 (i.e., experimental group) is better than that of the control group and other comparison groups.

Hereinafter, effects of the dynamic contact angle (DCA) of the silicone hydrogel lens having the water content of 50% are tested by using different experimental conditions of a concentration of the hyaluronic acid and PMB.

[Table 5-1] Experimental conditions of the storage solution and the dynamic contact angle of the silicone hydrogel contact lens having the water content of 50% of each of the lens groups are as follows. Specifically, the experimental conditions of the storage solutions 1 to 5 and the test results of the dynamic contact angle (DCA) are a same as those of the above-mentioned Table 1. Experimental conditions of each of storage solutions 6 to 9 are newly added, which include using the hyaluronic acid having a different molecular weight and a different concentration and PMB having a different concentration to test the effects of the dynamic contact angle (DCA) of the silicone hydrogel lens having the water content of 50%.

| | Added Amount in the Buffered Saline Solution (ppm) | | | | | | |
|---|---|---|---|---|---|---|---|
| | Low- | | High- | Ultra-high | | | |
| | molecular | Low-molecular | molecular | molecular | | | |
| | weight | weight | weight | weight | poly | | |
| | hyaluronic | hyaluronic | hyaluronic | hyaluronic | (MPC-co- | | Dynamic |
| | acid | acid | acid (Mw | acid (Mw | BMA) | | contact |
| Groups of | (Mw < 1,000 | (Mw~10,000 | 80-120k | 850-1600k | (i.e., | | angle |
| Experimental | Da) | Da) | Da) | Da) | PMB) | | (DCA) |
| Storage | — (No additional hydrophilic components added) | | | | | | 73.15° |
| solution 1 | | | | | | | |
| (control | | | | | | | |
| group) | | | | | | | |
| Storage | — | — | 600 ppm | — | — | | 53.20° |
| solution 2 | | | | | | | |
| (comparison | | | | | | | |
| group) | | | | | | | |
| Storage | 600 ppm | — | — | — | — | | 48.36° |
| solution 3 | | | | | | | |
| (comparison | | | | | | | |
| group) | | | | | | | |
| Storage | 600 ppm | — | — | — | 1,000 | ppm | 4.35° |
| solution 4 | | | | | | | |
| (experimental | | | | | | | |
| group) | | | | | | | |
| Storage | — | — | — | 600 ppm | 1,000 | ppm | 36.12° |
| solution 5 | | | | | | | |
| (comparison | | | | | | | |
| group) | | | | | | | |
| Storage | 100 ppm | — | — | — | 1,000 | ppm | 24.12° |
| solution 6 | | | | | | | |
| (experimental | | | | | | | |
| group) | | | | | | | |
| Storage | — | 600 ppm | — | — | 1,000 | ppm | 28.35° |
| solution 7 | | | | | | | |
| (experimental | | | | | | | |
| group) | | | | | | | |
| Storage | — | 100 ppm | — | — | 1,000 | ppm | 40° |
| solution 8 | | | | | | | |
| (experimental | | | | | | | |
| group) | | | | | | | |
| Storage | 600 ppm | — | — | — | 10,000 | ppm | 4.15° |
| solution 9 | | | | | | | |
| (experimental | | | | | | | |
| group) | | | | | | | |

Test results of Table 5-1 show that the storage solution including the low-molecular weight hyaluronic acid having a weight-average molecular weight that ranges from 1,000 Da to 10,000 Da and a concentration that ranges from 1,000 ppm to 10,000 ppm can enable the dynamic contact angle of the silicone hydrogel lens having the water content of 50% to have a relatively low value (e.g., less than 45 degrees).

Specifically, the storage solution 4 (i.e., experimental group) uses 600 ppm of the hyaluronic acid having a molecular weight of 1,000 Da and 1,000 ppm of PMB, and the storage solution 9 (i.e., experimental group) uses 600 ppm of the hyaluronic acid having a molecular weight of 1,000 Da and 10,000 ppm of PMB, which can have a dynamic contact angle of less than 5 degrees by testing, thereby showing a better wettability.

### Beneficial Effects of the Embodiments

In conclusion, in the liquid composition for contact lens and the contact lens product provided by the present disclosure, by virtue of “liquid composition for contact lens including a first hydrophilic component, a content of the first hydrophilic component being not greater than 1 wt %, the first hydrophilic component being a polymer having a phosphorylcholine group, and the first hydrophilic component having a first weight-average molecular weight of not less than 4,000 Da,” and “a content of the second hydrophilic component being not greater than 1 wt %, the second hydrophilic component being a hyaluronic acid or a salt thereof, and the second hydrophilic component having a second weight-average molecular weight of not greater than 10,000 Da,” a wettability of a surface of a contact lens can be greatly improved. In particularly, in the present disclosure, the liquid composition for contact lens can have a better wettability and a better moisturizing effect for trapping water of the surface of the contact lens than the conventional liquid composition for contact lens through an addition of the polymer having the phosphorylcholine group and a specific content of a low-molecular weight hyaluronic acid (or the salt thereof) in the liquid composition for contact lens.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A liquid composition for contact lens, comprising:
a first hydrophilic component, wherein a content of the first hydrophilic component is not greater than 1 wt %, the first hydrophilic component is a polymer having a phosphorylcholine group, and the first hydrophilic component has a first weight-average molecular weight of not less than 4,000 Da;
a second hydrophilic component, wherein a content of the second hydrophilic component is not greater than 1 wt %, the second hydrophilic component is a hyaluronic acid or a salt thereof, and the second hydrophilic component has a second weight-average molecular weight of not greater than 10,000 Da;
an inorganic salt, wherein a content of the inorganic salt is not greater than 5 wt %; and
a liquid water, wherein a content of the inorganic salt is not less than 80 wt %.

2. The liquid composition for contact lens according to claim 1, wherein the first hydrophilic component is selected from the group consisting of a homopolymer having the phosphorylcholine group and a copolymer having the phosphorylcholine group.

3. The liquid composition for contact lens according to claim 2, wherein the homopolymer having the phosphorylcholine group is poly(2-methacryloyloxyethyl phosphorylcholine), and the copolymer having the phosphorylcholine group is formed by a copolymerization of 2-methacryloyloxyethyl phosphorylcholine and a methacrylic acid ester, and wherein the methacrylic acid ester is an alkyl methacrylate having a C2 to C12 alkyl group or a hydroxypropyl methacrylate having a C2 to C12 hydroxyalkyl group.

4. The liquid composition for contact lens according to claim 1, wherein the first weight-average molecular weight of the first hydrophilic component ranges from 4,000 Da to 1,000,000 Da, the second weight-average molecular weight of the second hydrophilic component is less than the first weight-average molecular weight, and the second weight-average molecular weight is not greater than 5,000 Da.

5. The liquid composition for contact lens according to claim 4, wherein the first weight-average molecular weight of the first hydrophilic component ranges from 100,000 Da to 1,000,000 Da, and the second weight-average molecular weight is not greater than 3,000 Da.

6. The liquid composition for contact lens according to claim 1, wherein the second hydrophilic component is a sodium salt of hyaluronic acid or a potassium salt of hyaluronic acid.

7. The liquid composition for contact lens according to claim 6, wherein the second hydrophilic component is the sodium salt of hyaluronic acid, and the second weight-average molecular weight of the second hydrophilic component is not greater than 1,000 Da.

8. The liquid composition for contact lens according to claim 7, wherein the second hydrophilic component is a sodium hyaluronate having the second weight-average molecular weight or a hydrolyzed sodium hyaluronate.

9. The liquid composition for contact lens according to claim 1, wherein, based on a total weight of the liquid composition for contact lens being 100 wt %, a content of the first hydrophilic component ranges from 0.005 wt % to 1 wt %, a content of the second hydrophilic component ranges from 0.005 wt % to 1 wt %, a content of the inorganic salt ranges from 0.01 wt % to 5 wt %, and a content of the liquid water is not less than 90 wt %.

10. The liquid composition for contact lens according to claim 9, wherein the content of the first hydrophilic component ranges from 0.05 wt % to 1 wt %, and the content of the second hydrophilic component ranges from 0.01 wt % to 0.08 wt %.

11. The liquid composition for contact lens according to claim 1, wherein the first hydrophilic component has a first molality ranging from 1×10⁻⁸ mol/L to 2.5×10⁻⁴ mol/L, and the second hydrophilic component has a second molality ranging from 5×10⁻⁷ mol/L to 6×10⁻⁵ mol/L.

12. The liquid composition for contact lens according to claim 1, wherein the inorganic salt includes a chloride salt and a buffer salt, the chloride salt is selected from the group consisting of sodium chloride (NaCl), potassium chloride (KCl), and magnesium chloride (MgCl₂), and the buffer salt is selected from the group consisting of a boric acid or a salt thereof, a phosphoric acid or a salt thereof, a carbonic acid or a salt thereof, and citric acid or a salt thereof.

13. A contact lens product, comprising:
a packaging container;
the liquid composition for contact lens according to claim 1, wherein the liquid composition for contact lens is filled in the packaging container; and
a contact lens soaked in the liquid composition for contact lens, wherein the contact lens is a hydrogel contact lens or a silicone hydrogel contact lens.
